(19) United States
(12) Plant Patent Application Publication
(10) Pub. No.: US 2021/0037688 P1
(43) Pub. Date: Feb. 4, 2021

(54) DIANTHUS PLANT NAMED 'KLEDG18273'

(50) ;

(71) Applicant: Klemm+Sohn GmbH & Co. KG, Stuttgart (DE)

(72) Inventors: Nils Klemm, Stuttgart (DE); Lena Thiede, Bempflingen (DE)

(73) Assignee: Klemm+Sohn GmbH & Co. KG, Stuttgart (DE)

(21) Appl. No.: 16/915,217

(22) Filed: Jun. 29, 2020

Related U.S. Application Data
(60) Provisional application No. 62/922,263, filed on Jul. 30, 2019.

(51) Int. Cl. A01H 6/30

(52) U.S. Cl. PLT/272

(57) ABSTRACT

A new Dianthus plant named ‘KLEDG18273’ particularly distinguished by double flowers with a dark purple color and a magenta flame and a green-grey leaf color, is disclosed.

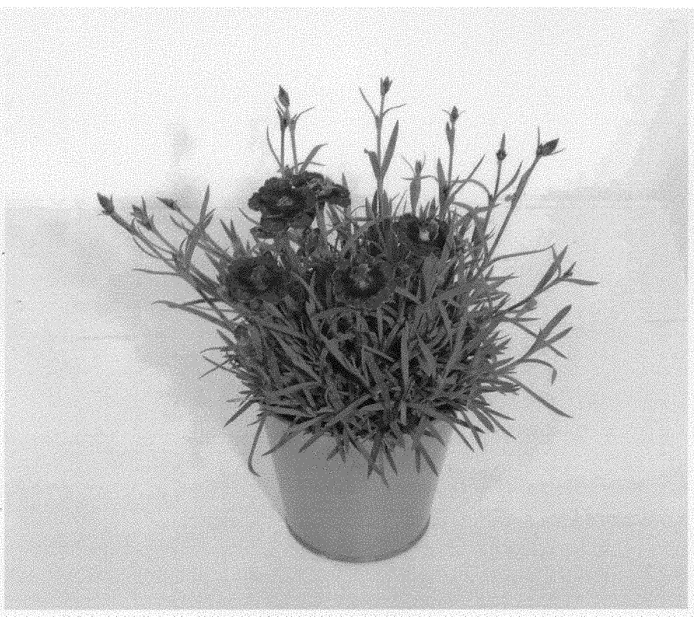

### CROSS REFERENCE TO RELATED APPLICATIONS

The present application is a non-provisional patent application and claims priority to U.S. Application No. 62/922,263, as filed Jul. 30, 2019, the entire contents of all which is herein incorporated by reference for all it may teach and disclose.

Genus and species: Dianthus sp.

Variety denominations: ‘KLEDG18273’.

### BACKGROUND OF VARIETY ‘KLEDG18273’

The present invention comprises a new and distinct variety of Dianthus, botanically known as Dianthus sp. and hereinafter referred to by the variety name ‘KLEDG18273’. ‘KLEDG18273’ is a selection that resulted from an open pollination of an unnamed and unpatented female dianthus line and an unknown male dianthus line.

In July of 2013, an unnamed female dianthus line and an unknown male dianthus line were cross pollinated in Stuttgart, Germany and seeds were obtained. The seeds were sown and plants were grown for evaluation. A plant line was selected in April of 2014 in Stuttgart, Germany, and named ‘KLEDG18273’. In May of 2014, ‘KLEDG18273’ was first vegetatively propagated by vegetative cuttings. ‘KLEDG18273’ was found to reproduce true to type in successive generations of asexual propagation via vegetative cuttings in Stuttgart, Germany and Latina, Italy.

### SUMMARY OF VARIETY ‘KLEDG18273’

The following are the most outstanding and distinguishing characteristics of this new variety when grown under normal horticultural practices in Latina, Italy.
1. Double flowers;
2. A dark purple color with a magenta flame; and
3. A green-grey leaf color.

### DESCRIPTION OF THE PHOTOGRAPH

This new Dianthus plant is illustrated by the accompanying photograph which show the plant's overall plant habit including form, foliage, and flowers of a 12-week-old plant grown in Stuttgart, Germany under greenhouse conditions in May 2015. The colors shown are as true as can be reasonably obtained by conventional photographic procedures.

### DESCRIPTION OF THE NEW VARIETY ‘KLEDG18273’

The following detailed descriptions set forth the distinctive characteristics of ‘KLEDG18273’. Data was collected on plants potted at eight weeks old in Stuttgart, Germany with the characteristics taken in June of 2019 in Latina, Italy. Color references are to The R.H.S. Colour Chart of The Royal Horticultural Society of London (R.H.S.), 5^{th} edition.
Classification:
   Family.—Caryophyllaceae.
   Species.—Dianthus sp.
   Common name.—Pot carnation.
   Parentage.—Female: An unnamed and unpatented dianthus line. Male: Unknown dianthus.
Propagation:
   Type.—Vegetative cuttings.
   Time to produce a rooted young plant.—14 to 18 days.
   Root description.—Young roots: RHS 161D. Mature roots: RHS 199C.
   Rooting habit.—Lanceolate.
Plant description:
   Plant form.—Upright.
   Plant height.—17 cm to 21 cm.
   Plant diameter or spread.—20 cm to 25.0 cm.
Lateral branches:
   Length.—5.0 cm to 10.0 cm.
   Diameter.—2.0 cm to 4.0 cm.
   Internode length.—2.5 cm to 4.0 cm.
   Number of internodes per branch/stem.—3 to 5.
   Node length.—0.2 cm to 0.4 cm.
   Node diameter.—0.2 cm to 0.4 cm.
   Texture.—Smooth.
   Color.—RHS 146C.
   Branch/stem.—Solid.
Foliage description:
   Arrangement.—Opposite.
   Length.—4.0 cm to 7.0 cm.
   Width.—0.3 cm to 0.5 cm.
   Shape.—Lanceolate.
   Apex.—Acute.
   Base.—Obtuse.
   Margin.—Entire.
   Texture.—Upper and lower surfaces: Smooth.
   Venetian pattern.—None.
   Venetian color.—None.
   Color.—Immature and mature leaves: Upper surface: RHS 146B. Lower surface: RHS 146C. Mature leaves: Both surfaces: RHS 191A.
Flower description:
   Flower type and habit.—Double flower, round.
   Fragrance.—Present.
   Natural flowering season.—Spring to fall.
   Position of flowers relative to the foliage.—Moderately.
   Number of flowers per spray.—3 to 4.
   Flower longevity on the plant.—14 to 16 days.
   Flower buds.—Length: 1.5 cm to 2.0 cm. Diameter: 0.4 cm to 0.6 cm. Shape: Elliptic. Color: RHS 147B. Extrusion of the styles at the bud stage: Absent.
   Corolla.—Quantity of petals/arrangement: 10 to 14. Corolla height: 0.8 cm to 1.5 cm. Petal length: 3.0 cm to 3.5 cm. Petal width: 1.2 cm to 1.6 cm. Petal shape: Spoon shaped. Petal apex: Obtuse. Petal base: Acute. Petal margin: Serrated. Depth of petal margin: Medium. Petal texture: Upper and lower surfaces: Smooth. Petal color: Immature: Upper surface: RHS 79A and flamed RHS 80B. Lower surface: RHS 80D and outer border RHS 80B. Mature: Upper surface: RHS 79A and flamed RHS 80B. Lower surface: RHS 80D and outer border RHS 80B. Calyx: Quantity of sepals/arrangement: Composed of 5 sepals. Shape: Campanulate. Calyx height: 1.5 cm to 2.0 cm. Calyx diameter, at the apex: 0.3 cm to 0.5 cm. Sepal texture: Upper and lower surfaces: Smooth. Sepal color: Upper surface: RHS 137A. Lower surface: RHS 137D.
Peduncles:
   Length.—1.0 cm to 2.0 cm (last internode), whole stem 10.0 cm.
   Diameter.—0.1 cm to 0.2 cm.
   Strength.—Moderate.
   Texture.—Smooth.
   Color.—RHS 191A.
Reproductive organs:
   Stamens.—Quantity: 4 to 8. Filament length: 1.5 cm to 2.0 cm. Filament color: RHS 157B. Anther length: 0.2 cm to 0.3 cm. Anther diameter: 0.1 cm. Anther color: RHS 85C. Pollen amount: Moderate.
   Pistils.—Quantity: 2 styles. Pistil length: Not observed. Style length: 1.0 cm to 2.0 cm. Style color: RHS 155D. Stigma shape: Filamentous. Stigma color: RHS 85C. Ovary size: 0.5 cm to 0.7 cm. Ovary shape: Oval. Ovary color: RHS 145C. Fruits/seeds: Seed sometimes observed.
Disease and insect/pest resistance: No special observations were made

### COMPARISON OF ‘KLEDG18273’ WITH KNOWN VARIETY When ‘KLEDG18273’ is compared to the commercial line ‘KLEDG18305’ (U.S. Application No.: 15/998,600), the following differences are noted in Table 1:

TABLE 1
Comparison with Similar Variety

| | Variety | Commercial line |
|---|---|---|
| Trait | ‘KLEDG18273’ | ‘KLEDG18305’ |
| Flower type | Double | Single |
| Flower color | Dark purple, magenta flamed | Pink with purple ring |
| Leaf color | Green-grey | Dark green |

We claim:

1. A new and distinct variety of Dianthus plant named ‘KLEDG18273’ as shown and described herein.
